(19) United States
(12) Patent Application Publication
(10) Pub. No.: US 2013/0296630 A1
(43) Pub. Date: Nov. 7, 2013

(54) Systems and Method for Treatment of Tumors

(71) Applicant: Amie B. Franklin, Woodbury, MN (US)

(72) Inventor: Amie B. Franklin, Woodbury, MN (US)

(21) Appl. No.: 13/464,183

(22) Filed: May 4, 2012

(51) Int. Cl. A61M 37/00; A61M 36/04

(52) U.S. Cl. 600/3; 604/23

(57) ABSTRACT

An oxygen-producing device has a power supply with an electrically positive and an electrically negative output, at least one pair of electrodes, one electrode of the pair coupled to the electrically positive output and the other coupled to the electrically negative output, an oxygen-rich material exposed to the electrodes for producing oxygen in response to a voltage generated across the electrodes, and an envelope containing the power supply, the pair of electrodes and the oxygen rich material. The device operates within a human tumor, the envelope— comprises a gas-permeable membrane for releasing the produced oxygen into an environment outside of the envelope and the power supply is enabled to provide a DC voltage of at least 1.2 volts to the electrodes.

### CROSS-REFERENCE TO RELATED DOCUMENTS

N/A

### BACKGROUND OF THE INVENTION

1. Field of the Invention

The present invention relates to methods and devices for treatment of cancerous tissues, and more particularly to such devices producing oxygen in treatment.

2. Discussion of the state of the art.

Without oxygen, pretty much all of today's anti-cancer weapons are useless. Feeding the tumor may actually be better for the patient than starving the tumor of oxygen. “It's like a two-headed beast,” says Edith Lord, Ph.D., professor of Oncology in Microbiology & Immunology at the University of Rochester's cancer center. “If you cut off the blood vessels, the tumor doesn't grow, but its also harder to treat with current therapies.”

Five years ago saw the dawn of a new era in cancer research—the pursuit of anti-angiogenesis, or the cutting off or prevention of blood vessel growth. This was hailed as a new way to knock out tumors by starving them of oxygen. But progress has been slow and spotty, and scientific results inconsistent. There have been a few clinical trials of the new medicines, but none is yet approved for widespread use.

Now doctors are coming more to terms with the negative complications of starving tumors of oxygen. “The crucial role that oxygen plays in killing tumors has been under appreciated,” says Bruce Fenton, Ph.D., associate professor of radiation oncology at the Wilmot Cancer Center. Radiation and other current therapies rely on the formation of harmful molecules known as free radicals to damage cells, but without oxygen their efforts fall short as cells can often repair themselves. Cancer cells that contain oxygen are about two to three times more vulnerable to radiation than cells without, says Fenton.

Colleague Paul Okunieff, M.D., head of Radiation Oncology at the Wilmot Cancer Center, is more blunt about the effects of low oxygen, known as hypoxia. “The tumor is meaner if it's hypoxic,” Okunieff says, “Oxygen is by far the most powerful molecule for making cells vulnerable to radiation. Tumor cells that survive hypoxic conditions are often the cells that are most aggressive, most hardy, and most likely to go out and start new cancer colonies,” he says. They're also the tumor cells most likely to have mutations that make them prone to spreading.

For decades scientists have tried an opposite approach, by feeding oxygen to tumors to kill them more effectively. Doctors have asked patients to breathe extra oxygen during radiation treatments to make tumors more vulnerable to radiation; they've given patients transfusions so there would be more oxygen-carrying red blood cells in tumors; and they've tried other methods to take advantage of oxygen's killing abilities.

While some methods have had some success, none has worked well consistently, says Okunieff. Meanwhile, with a surge of anti-angiogenesis research, researchers continue to study the consequences of starving the tumor of oxygen.

“Those areas of low oxygen in tumors are more resistant to our treatments,” says Lord, “for a number of reasons.” Besides less oxygen to form free radicals, cells under low-oxygen conditions don't divide as much, so they have more time to repair themselves before being vulnerable to radiation and other measures that target dividing cells. It's also harder to get drugs to areas without blood vessels, and without those blood vessels even the body's natural cancer-fighting, immune cells can't reach the tumor to attack it.

“Tumors that have insufficient oxygen tend to be more likely to spread from the primary site to other parts of the body.” Michael Weber, director of U.Va.'s Cancer Center. “Despite the overall importance of tumor hypoxia, it is very difficult to measure directly and most methods that are available are very expensive.”

### SUMMARY OF THE INVENTION

In an embodiment of the present invention an oxygen-producing device is provided, comprising a power supply with an electrically positive and an electrically negative output, at least one pair of electrodes, one electrode of the pair coupled to the electrically positive output and the other coupled to the electrically negative output, an oxygen-rich material exposed to the electrodes for producing oxygen in response to a voltage generated across the electrodes, and an envelope containing the power supply, the pair of electrodes and the oxygen rich material. The device operates within a human tumor, the envelope—comprises a gas-permeable membrane for releasing the produced oxygen into an environment outside of the envelope and the power supply is enabled to provide a DC voltage of at least 1.2 volts to the electrodes.

In one embodiment the power supply comprises a battery rechargeable by magnetic induction. Also in one embodiment the power supply comprises a pair of cantilevered strips coupled to the electrodes, one of which strips is composed at least in part of a radioactive material that emits electrons, and the other is a metal strip, such that electrons emitted from the first strip collect on the second strip producing an electrical potential that appears across the electrodes. In some embodiments at least one of the strips is of a flexibility that the opposite charges cause the flexible strip to deflect toward the other strip, resulting over time in a proximity of the strips that results in discharge of the potential, after which the flexible strip returns to the un-deflected state, and the charge begins to rebuild.

In one embodiment the envelope includes a port in lieu of the membrane for discharge of gases from within the envelope. The oxygen-rich material may be water or hydrogen peroxide.

In another aspect of the invention a method for treating a tumor with oxygen is provided, comprising the steps of (a) fashioning an oxygen-producing device, comprising a power supply with an electrically positive and an electrically negative output, at least one pair of electrodes, one electrode of the pair coupled to the electrically positive output and the other coupled to the electrically negative output, an oxygen-rich material exposed to the electrodes for producing oxygen in response to a voltage generated across the electrodes, and an envelope containing the power supply, the pair of electrodes and the oxygen rich material, the device no more than 25 mm in any dimension; and (b) inserting the device into or near a tumor in a human body.

In one embodiment the envelope—comprises a gas-permeable membrane for releasing the produced oxygen into an environment outside of the envelope and the power supply is enabled to provide a DC voltage of at least 1.2 volts to the electrodes. Also in one embodiment the power supply comprises a battery rechargeable by magnetic induction. The power supply may comprise a pair of cantilevered strips coupled to the electrodes, one of which strips is composed at least in part of a radioactive material that emits electrons, and the other is a metal strip, such that electrons emitted from the first strip collect on the second strip producing an electrical potential that appears across the electrodes. Also, at least one of the strips may be of a flexibility that the opposite charges cause the flexible strip to deflect toward the other strip, resulting over time in a proximity of the strips that results in discharge of the potential, after which the flexible strip returns to the un-deflected state, and the charge begins to rebuild.

IN some embodiments the envelope includes a port in lieu of the membrane for discharge of gases from within the envelope. Also in some embodiments the oxygen-rich material is water or hydrogen peroxide.

### BRIEF DESCRIPTION OF THE DRAWING FIGURES

FIG. 1 is an illustration of a tumor, and comparative size of an apparatus of the invention for providing oxygen to the tumor.

FIG. 2 is an illustration of an apparatus for supplying oxygen to a tumor according to one embodiment of the present invention.

FIG. 3 is an illustration of an apparatus as in FIG. 2, including a novel power supply in an embodiment of the invention.

FIG. 4 illustrates an alternative embodiment of the present invention.

### DETAILED DESCRIPTION OF THE INVENTION

The present patent application teaches and claims the use of Oxygen and other gas devices for treatment of tumors. This includes devices that can be implanted, injected, surgically sewn in, applied to an open wound, etc. that provide or increase oxygen in the tumor or cancer. This might be with heat and or cold, cryo, or radiation, electrical stimulation, battery (electrolosis), combined with chemo, alloderm (tissue flaps), implants, biologics, UV, mechanical, stem cell or other ways of treating tumors and or cancer. Treatments used in cancer medicine rely upon an increase in oxygen in the tumor or produce an increase in oxygen. When the oxygen is decreased in the tumor the therapy may not work.

It is well understood that tumors, having insufficient oxygen, tend to be more likely to spread from the primary site to other parts of the body. Despite the overall importance of tumor hypoxia, it is very difficult to measure directly and most methods that are available are very expensive.

Doctors have asked patients to breathe extra oxygen during radiation treatments to make tumors more vulnerable to radiation; they've given patients transfusions so there would be more oxygen-carrying red blood cells in tumors; and they've tried other methods to take advantage of oxygen's killing abilities. The crucial role that oxygen plays in killing tumors has not been appreciated, and is a focus of methods and devices taught in this patent application. Radiation and other current therapies rely on the formation of harmful molecules known as free radicals to damage cells, but without oxygen their efforts fall short, as cells can often repair themselves. Cancer cells that contain oxygen are about two to three times more vulnerable to radiation than cells without.

The tumor is meaner if it's hypoxic. Oxygen is by far the most powerful molecule for making cells vulnerable to radiation. Tumor cells that survive hypoxic conditions are often the cells that are most aggressive, most hardy, and most likely to go out and start new cancer colonies. They're also the tumor cells most likely to have mutations that make them prone to spreading. An object of the present invention is to produce an environment in the cancerous tissues that is enhanced in free oxygen, that is oxygen-rich, or at least more so than the typical (morbid or pathological) surrounding tissues.

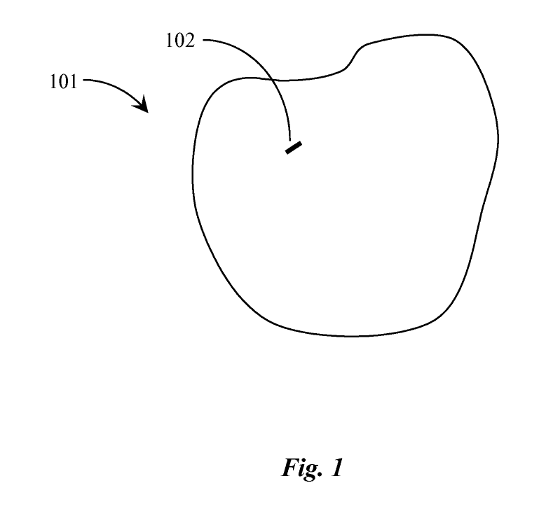

An object of the present invention is to produce an environment in a tumor that is enhanced in free oxygen, that is oxygen-rich, or at least more so then the typical (morbid or pathological) content of the tumor. FIG. 1 is a simplistic illustration of a tumor in a human. The skilled artisan will understand that tumors occur, and are treated, that vary in size from minute to quite large, say from a fraction of a centimeter to several centimeters or more in diameter or thickness. I an embodiment of the present invention a small device 102 is provided of a size that may be easily injected into a tumor, or in some cases may be injected near a tumor, to generate oxygen to which the tumor will be exposed. FIG. 1 is intended to just provide context for the sizes that might be involved.

The tumor 101 in FIG. 1 is perhaps two centimeters in thickness. For a device to be easily planted in such a tumor the device needs to be quite small, such that may be injected via a syringe, or implanted by a needle thrust carrying the device, doing minimal damage to surrounding tissue. Sizes and dimensions can vary, but the devices illustrated and described herein will typically be of a size that would occupy an envelope no more than two or three millimeters on a side, and in most cases smaller yet.

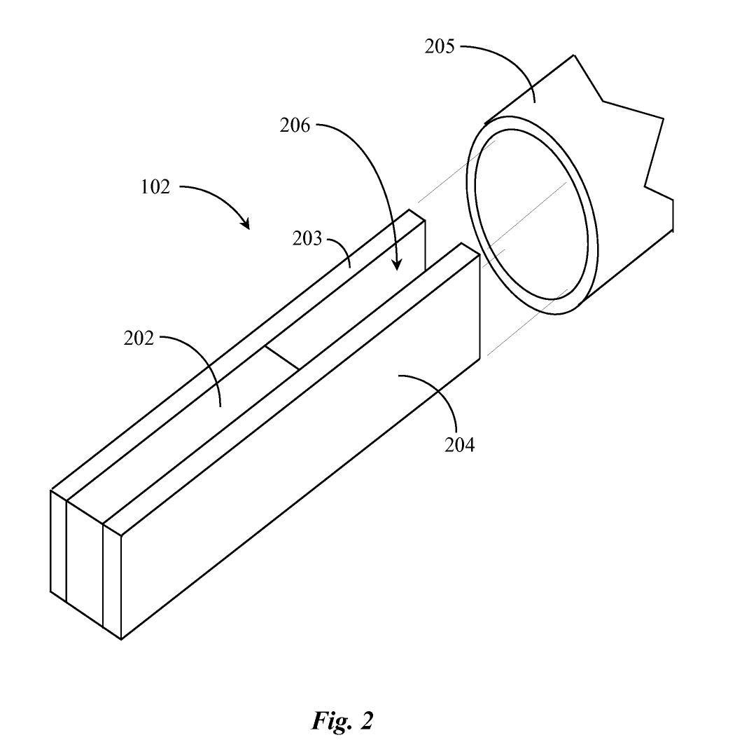

FIG. 2 is an illustration of a device 102 in one embodiment the present invention that produces oxygen by electrolysis (traditionally used to produce hydrogen) and can be implanted in a tumor. Device 102 comprises a power supply 202, and a pair of electrodes 203 and 204, in which the electrodes contact the body of the power supply along opposite sides of the body, and extend for a distance beyond the body, providing a space 206 between the electrodes. Tubing 205 represents the action end of a surgical needle, and the cross-section of the power supply and electrodes is of a size that the assembly in one direction may pass through the eye of the needle or canulae or in a solution through either. In another embodiment the device may have an interface for attaching in one direction to a needle, so the device may be carried through tissue with a needle thrust, and will remain behind when the needle is withdrawn.

The illustration of an end of a surgical needle in FIG. 2 is not to suggest that a device 102 may be aspirated into a syringe, and then injected along with a fluid into a tumor, although this means of placement might in some cases be useful. A preferable system for placement would use a portion of a surgical needle, but a device 102 would be placed in the hollow shaft of the needle portion, close to the end, under a microscope or other magnification apparatus. The needle portion comprises a rod in the hollow shaft that may be manipulated. After placement of the device 102, the needle portion is dipped in perhaps a saline solution, or another antiseptic, anti-bacterial fluid, the needle is inserted into the tumor to be treated, with the point of the needle placed at the point in the tumor where the device is desired to be placed, then the device 102 is pushed out of the needle by translating the rod forward, pushing the device 102 out of the needle and into the tumor. The needle is then withdrawn. (If the size is 2-3 mm then it would be more properly called a cannula than a needle.)

Electrodes 203 and 204 in this embodiment are metal film, and power supply body 202 is of a non-electrically-conductive material, which may be ceramic, such that electrodes 203 and 204 are never electrically shorted. Output contacts on each side of power supply 202 make contact with electrodes 203 and 204 in such a manner that a voltage may be induced across electrodes 203 and 204. blood or other liquid matter in the tumor when device 102 is implanted in the tumor, will fill space 206, and voltage induced across the electrode gap will disassociate water in the liquid into oxygen and hydrogen.

Both the oxygen and the hydrogen produced will be taken into solution in the liquid matter in the tumor. In some cases liquid, such as saline solution, may be injected with the device to ensure adequate water at the desired point in the tumor to produce the needed oxygen. At least a portion of the oxygen will be available as therapeutic material for treatment of the tumor.

In this embodiment of apparatus 102, water from the aqueous environment in the tumor is electrolyzed to form oxygen by applying a voltage of 1.2 volt DC minimum potential across electrodes 203 and 204 of the device by power supply 202. The reaction produced in electrolysis is 2H₂0−>2H₂+O₂. The hydrogen byproduct from electrolysis is eliminated by absorption into the bloodstream of the individual.

In some treatment procedures more than one oxygen generator of the sort described may be injected into a tumor to maximize and/or distribute production of oxygen, and multiple devices may be placed at different positions to provide oxygen generation over a planned region.

Power supply 202 in some embodiments may comprise an onboard battery system, which in some embodiments might be a rechargeable system responsive to an ambient alternating magnetic field inductively producing an electric current in a charging circuit coupled to power supply 202.

In an alternative embodiment a bio-based micro-battery might be used as well. Additionally, the apparatus might also be powered by a miniaturized nickel-based nuclear cell, which could provide power consistently for an indefinite period. The bio-battery in one embodiment might be based on synthetic ion transport proteins. Ionic species of a gas may be produced at one electrode, where the produced ionic species is transported across an ion-permeable membrane, and the transported ionic species react at a second electrode to be converted into a molecule which produces a net change of concentration of oxygen.

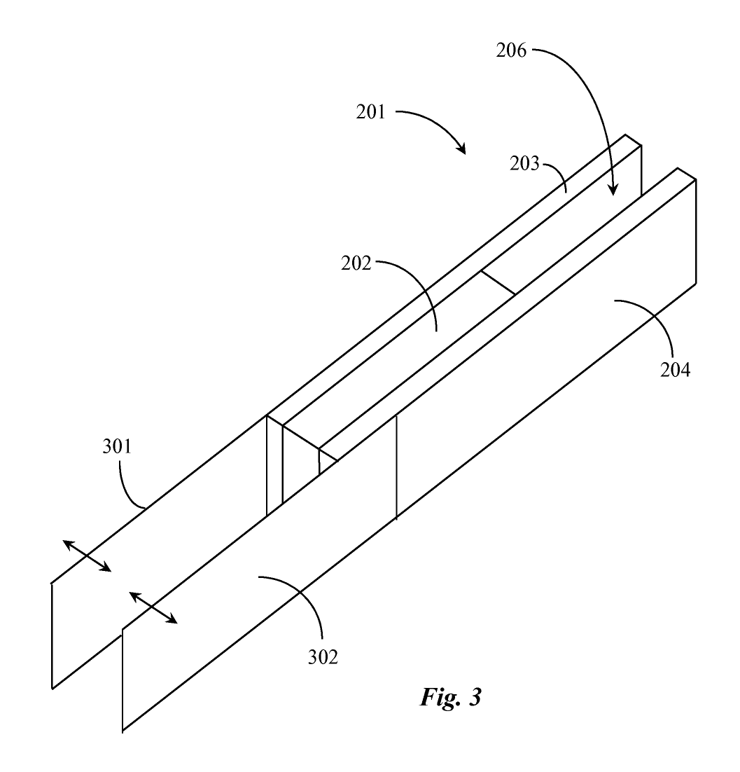

FIG. 3 illustrates schematically a nickel-based nuclear cell, as briefly described above, integrated to a pair of electrodes as described with reference to FIG. 2. In this case element 202 is a simply a spacer for insulating the two electrodes from one another electrically so an electrical potential may be established across the electrodes. A metal strip 301, which may be about one millimeter wide, two millimeters long and 60 micrometers (millionths of a meter) thick is spaced apart from a thin film of radioactive nickel-63, which emits beta particles (electrons). The emitted electrons collect on the metal strip, which may be, for example, copper, producing a negative charge (buildup of excess electrons with no path for an electrical current). The isotope film, losing electrons, becomes positively charged relative to the opposed metal strip.

The metal strip 301, that becomes negatively charged, is congruent with electrode 203, and the electron-emitting strip 302 is congruent with electrode 204. Therefore the voltage induced between metal strip 301 and strip 302 appears also across electrodes 203 and 204. As the voltage builds up, strips 301 and 302, cantilevered as shown, deflect toward one another until at a close approach a current flows and the charge is dissipated. As soon as the charge dissipates, the voltage begins to build again.

The spacing and geometry is controlled to produce a voltage that exceeds 1.2 volts D.C. considered a minimum to drive electrolysis of water to produce oxygen under these conditions. The voltage may be allowed to build to somewhat above 1.2 volts in operation. The net result is an oscillating voltage across electrodes 203 and 204, which produces electrolysis for the time that the voltage exceeds 1.2 volts D.C.

In some cases the thickness of the strips is controlled such that only one (say the metal strip 301) will deflect significantly under the voltage influence. Also, in some cases there need not be separate electrodes, as the metal strip and the radioactive nickel strip serve also as the electrodes for electrolysis.

In yet another embodiment the battery might be a tiny solar panel, which might in some embodiments be implemented as a contact lens, or power might be provided by a solar-powered chip.

Also in an alternative embodiment oxygen might be supplied in the tumor by a solar powered nano-motor that could produce oxygen. In such a motor absorption of sunlight by one of two stoppers, a light-harvesting one, causes transfer of one electron to a station A, which is deactivated as far as wanting a ring to encircle it. As a consequence, the ring moves to its second port of call, station B. Station A is subsequently reactivated by the return of the transferred electron to the light-harvesting stopper, and the ring moves back to this station.

In some cases an oxygen generator as described above may generate oxygen substantially without generating free hydrogen using a multilayer electrolyzer sheet having a proton exchange membrane sandwiched by an anode layer and a cathode layer.

Radioactive isotopes can continue to release energy over periods ranging from weeks to decades. The half-life of nickel-63, for example, is over 100 years. So a device thus powered might provide oxygen to a tumor for a long period of time.

A moving cantilever may also directly actuate a linear device or can move a cam or ratcheted wheel to produce rotary motion. A magnetized material attached to the rod can generate electricity as it moves through a coil. Or a nano battery which incorporates covering the electrodes with millions of tiny filaments called nanotubes. Each nanotube is 30,000 times thinner than a human hair. Nanotube filaments increase the surface area of the electrodes and provide enhanced capacitance to store more energy. The nano battery, which has the longevity of present battery technology, has the speed of a capacitor. Or a battery may consist of a miniaturized galvanic cell comprising a cathode, anode, and electrolyte.

In another aspect of the invention water may be supplied with the device 201 and may be stored with the device and consumed from its own reservoir. This reservoir could be refillable or single use. Advantages of supplying water would be that you wouldn't disturb the balance/ratio of other ingredients suspended in fluid of a tumor, and prevent contamination of the electrodes.

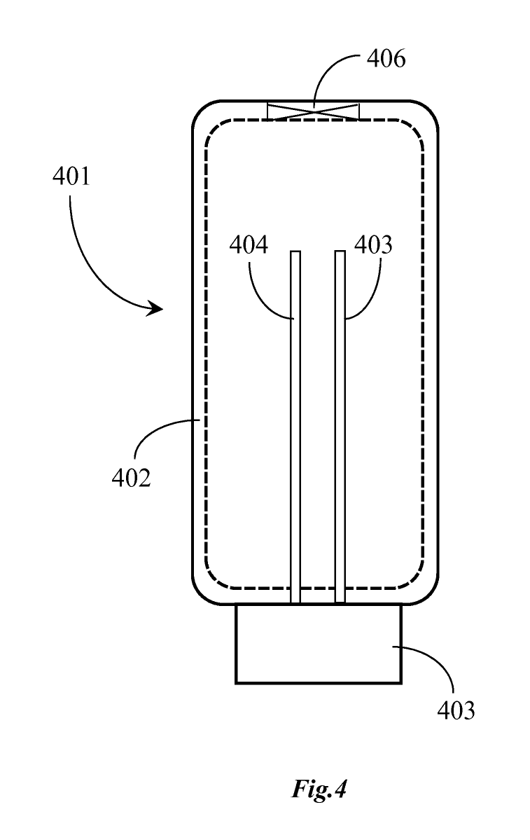

FIG. 4 illustrates schematically a water-filled capsule 401 having electrodes 402 and 403 spaced apart in fluid inside the capsule. Capsule 401 comprises a glass envelope 402 in this embodiment, although the envelope might be of other benign material in other embodiments. Electrodes 403 and 404 seal through glass envelope 402, and connect to a power supply 403, which may be a power supply of any nature described for the purpose in this specification, such as a miniature battery, a nuclear cell, and so forth. At least one region of envelope 402 comprises a permeable membrane 406, which is a hydrophobic but gas-permeable membrane that lets gas out but keeps the liquid in. Also in some embodiments the exit of gas from the envelope might be through a one-way valve that might be accomplished by a pierced flexible polymer material, a needle hole in a rubber plug by analogy.

In one embodiment the liquid with which the capsule is filled is water, and oxygen is produced by electrolysis, as described above, and escapes, along with the hydrogen produced, through the membrane or one-way valve. It should be noted that the gases produced by electrolysis create a pressure in the capsule, but gases are very quickly absorbed in solution by the water. The capsule can be expected under a steady-state operation to expel some fluid, due to the increased pressure, the fluid being oxygen rich. When the operation ceases, due to either being switched off or the power supply failing, the pressure should return to equilibrium with the surrounding environment. Oxygen produced will be expected to interact in the tumor in beneficial ways, but likely not all of the oxygen produced will be so employed. Much may simply be removed through the natural action of the fluids with the blood supply. Hydrogen produced will also be removed over time by the same mechanism in many embodiments.

In some embodiments one might use various techniques to release the water from a pre-filled reservoir to the electrolyzing electrodes at just a desired rate, or it could be rate controlled by using the water supply controller as a preset or remotely controlled selectable level.

In some embodiments another oxygen-rich liquid might be used instead of or in conjunction with water in such a capsule. Hydrogen peroxide is one such material. The bonding energies for some oxygen-rich liquids are less than that for water, and might be expected to electrolyze to produce free oxygen more readily than water.

In yet another aspect of the invention water enriched with oxygen-filled nanobubbles could be used either alone or in conjunction with another device to provide oxygen in the tumor. Such water could augment or complement any device providing oxygen such as the devices described herein. In some case enriched water may be provided along with implanting a device according to the invention.

In other aspects of the present invention injectable hyaluronic acid or nano-packaged hydrogen peroxide with a bio compatible catalase or enzyme/catalyst that releases O₂ on a renewable basis may be employed. This or some other chemical reaction such as H₂O₂ could be encapsulated in Hyaluronic Acid or a bio compatible nano encapsulation. Both are devices capable of generating O₂ from chemical reactions, to deliver oxygen into the tumor, and an envelope much like that of FIG. 4 might be used, in some cases without electrodes and power supply, and in some cases with.

In systems which produce hydrogen as a by-product, such as electrolytic systems, the system may include venting mechanisms to eliminate hydrogen. In systems, which use air as a source for electrochemical extraction of oxygen, an inlet port may be included

In another aspect injectable hyaluronic acid (HA) or other relatively inert agents may be used as a carrier or delivery vehicle of oxygenated material for oxygenating the interior of the tumor. For example, using a nano-bead with a slow diffusion gradient, oxygen may be diffused into the tumor over a 90 day period.

It will be apparent to the skilled artisan that many alterations may be made to the embodiments described herein without departing from the spirit and scope of the invention. Sizes and materials may vary. Means of injection and implantation may vary. Different oxygen-producing materials may be used, all within the spirit and scope of the invention. The invention is to be defined, therefore, only by the scope of the claims which follow:

I claim:

1. An oxygen-producing device, comprising:
a power supply with an electrically positive and an electrically negative output;
at least one pair of electrodes, one electrode of the pair coupled to the electrically positive output and the other coupled to the electrically negative output;
an oxygen-rich material exposed to the electrodes for producing oxygen in response to a voltage generated across the electrodes; and
an envelope containing the power supply, the pair of electrodes and the oxygen rich material;
wherein the device operates within a human tumor, the envelope—comprises a gas-permeable membrane for releasing the produced oxygen into an environment outside of the envelope and the power supply is enabled to provide a DC voltage of at least 1.2 volts to the electrodes.

2. The device of claim 1 wherein the power supply comprises a battery rechargeable by magnetic induction.

3. The device of claim 1 wherein the power supply comprises a pair of cantilevered strips coupled to the electrodes, one of which strips is composed at least in part of a radioactive material that emits electrons, and the other is a metal strip, such that electrons emitted from the first strip collect on the second strip producing an electrical potential that appears across the electrodes.

4. The device of claim 3 wherein at least one of the strips is of a flexibility that the opposite charges cause the flexible strip to deflect toward the other strip, resulting over time in a proximity of the strips that results in discharge of the potential, after which the flexible strip returns to the un-deflected state, and the charge begins to rebuild.

5. The device of claim 1 wherein the envelope includes a port in lieu of the membrane for discharge of gases from within the envelope.

6. The device of claim 1 wherein the oxygen-rich material is water or hydrogen peroxide.

7. A method for treating a tumor with oxygen, comprising the steps of:
(a) fashioning an oxygen-producing device, comprising a power supply with an electrically positive and an electrically negative output, at least one pair of electrodes, one electrode of the pair coupled to the electrically positive output and the other coupled to the electrically negative output, an oxygen-rich material exposed to the electrodes for producing oxygen in response to a voltage generated across the electrodes, and an envelope containing the power supply, the pair of electrodes and the oxygen rich material, the device no more than 25 mm in any dimension; and
(b) inserting the device into or near a tumor in a human body.

8. The method of claim 7 wherein the envelope—comprises a gas-permeable membrane for releasing the produced oxygen into an environment outside of the envelope and the power supply is enabled to provide a DC voltage of at least 1.2 volts to the electrodes.

9. The method of claim 7 wherein the power supply comprises a battery rechargeable by magnetic induction.

10. The method of claim 7 wherein the power supply comprises a pair of cantilevered strips coupled to the electrodes, one of which strips is composed at least in part of a radioactive material that emits electrons, and the other is a metal strip, such that electrons emitted from the first strip collect on the second strip producing an electrical potential that appears across the electrodes.

11. The method of claim 10 wherein at least one of the strips is of a flexibility that the opposite charges cause the flexible strip to deflect toward the other strip, resulting over time in a proximity of the strips that results in discharge of the potential, after which the flexible strip returns to the un-deflected state, and the charge begins to rebuild.

12. The method of claim 7 wherein the envelope includes a port in lieu of the membrane for discharge of gases from within the envelope.

13. The method of claim 7 wherein the oxygen-rich material is water or hydrogen peroxide.
